(19) United States
(12) Patent Application Publication
(10) Pub. No.: US 2020/0411186 A1
(43) Pub. Date: Dec. 31, 2020

(54) ACNE INTELLIGENCE DIAGNOSTIC AND PRODUCT RECOMMENDATION MOBILE APP

(71) Applicant: SkinClinical AI, LLC, Chicago, IL (US)

(72) Inventors: Mark Blanchard, Atlanta, GA (US); Niv Carmi, San Diego, CA (US); Jonathan Flicker, Locust Valley, NY (US)

(21) Appl. No.: 16/911,582

(22) Filed: Jun. 25, 2020

Related U.S. Application Data
(60) Provisional application No. 62/866,075, filed on Jun. 25, 2019.

(51) Int. Cl. G16H 50/20; G16H 20/30; G06T 7/00

(52) U.S. Cl. CPC G16H 50/20; G16H 20/30; G06T 2207/30088; G06T 2207/30201; G06T 7/0012

(57) ABSTRACT

An Acne Intelligence mobile app provides acne sufferers with a smart phone-based platform that combines Computer Vision and Artificial Intelligence to analyze the skin periodically, therefore addressing the importance of understanding the needs and specific circumstances of each consumer. The analysis is combined with an algorithm that recommends and enables the purchase of specific products that best address the results from the analysis.

### CROSS-REFERENCES TO RELATED APPLICATIONS

This application claims the benefit of U.S. Provisional Patent Application No. 62/866,075, filed Jun. 25, 2019, the entire content of which is herein incorporated by reference.

### STATEMENT REGARDING FEDERALLY SPONSORED RESEARCH OR DEVELOPMENT

(NOT APPLICABLE)

### BACKGROUND

Consumer acne treatments are for the most part one-size-fits-all and often generate short-term results at best. When leading treatments fail to provide longer lasting results, consumers often consult their dermatologist or other skin care specialists who can prescribe antibiotics or controversial drugs. In the cases where acne specialists can provide a more tailored approach to consumers, the costs associated with frequent consultations quickly become prohibitive for a great number of acne sufferers. Ultimately, these consumers have no choice but to switch from one generic solution to the next.

### SUMMARY

The Acne Intelligence mobile app resolves this situation from different perspectives. First, it provides acne sufferers with a free smart phone-based platform that combines Computer Vision and Artificial Intelligence to analyze the skin periodically, therefore addressing the importance of understanding the needs and specific circumstances of each consumer; and second, by combining this analysis with a proprietary algorithm that recommends and enables the purchase of specific products that best address the results from the analysis.

Moreover, this analysis is performed on a weekly basis to monitor progress. Over a fixed period of time, e.g., every four weeks, the app will determine the most effective combination of products based on this progress, which will be shipped to the consumer for use during the next cycle.

Importantly, the app will learn and further the efficacy of the system through Artificial Intelligence as more data becomes available.

The acne intelligence diagnostic and product recommendation mobile app combines Computer Vision and Artificial Intelligence to analyze the skin, coupled with a proprietary product recommendation algorithm designed to recommend optimal/customized products to treat acne effectively. The algorithm accounts for every possible combination of results from the analysis and assigns a combination of products that is specific and most adequate for the user's skin conditions.

The user enrolls in a subscription and the products are purchased through the app. They are grouped into AM and PM routines that last for a predetermined cycle (e.g., 4 weeks). The analysis is performed weekly and toward the end of the cycle (e.g., on the third week in a 4-week cycle), based on the results from that cycle's analysis, a new set of products is defined and shipped, to arrive in time to start the next cycle.

### BRIEF DESCRIPTION OF THE DRAWINGS

These and other aspects and advantages will be described in detail with reference to the accompanying drawings, in which:

FIGS. 1 and 1(a)-(d) show the general sequence/flow of screens for the app.

FIG. 2 illustrates sequence of screens during skin analysis.

FIGS. 3(a)-(f) show the algorithms that determine which products are recommended.

FIG. 4 illustrates the screen where the recommended AM/PM routines are described after the analysis.

FIG. 5 illustrates the screen where the specific products that make up the routines are being recommended for purchase.

FIG. 6 represents an example of a skin analysis with multiple (e.g., 21) regions.

### DETAILED DESCRIPTION

The process begins with the marketing methods that are used to introduce the app to the target audience with the objective of generating downloads and ultimately subscriptions to the system. These marketing methods are direct to consumer based and consist mostly of paid advertising through social media networks to the target audience, relevant decision makers and influencers. The content of the advertising emphasizes the unique differentiation points as being a technology focused, customized solution for a widespread and common skin condition: acne.

Viewers of the advertising are able to click the appropriate links to download the app directly to their phones, or have the option of visiting the acne intelligence website to consume additional information before downloading the app.

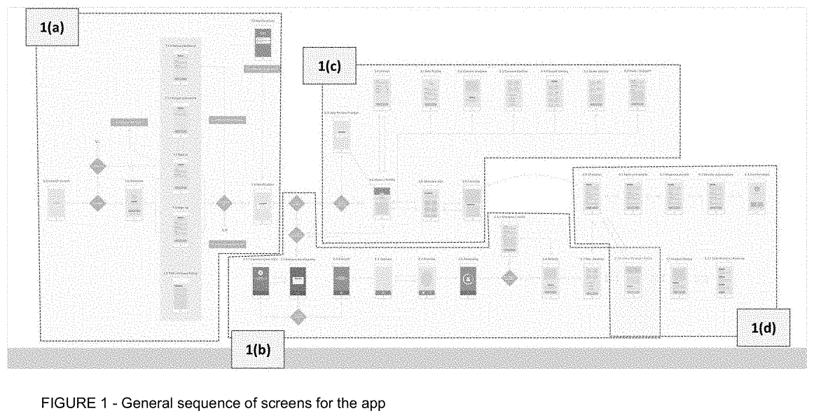

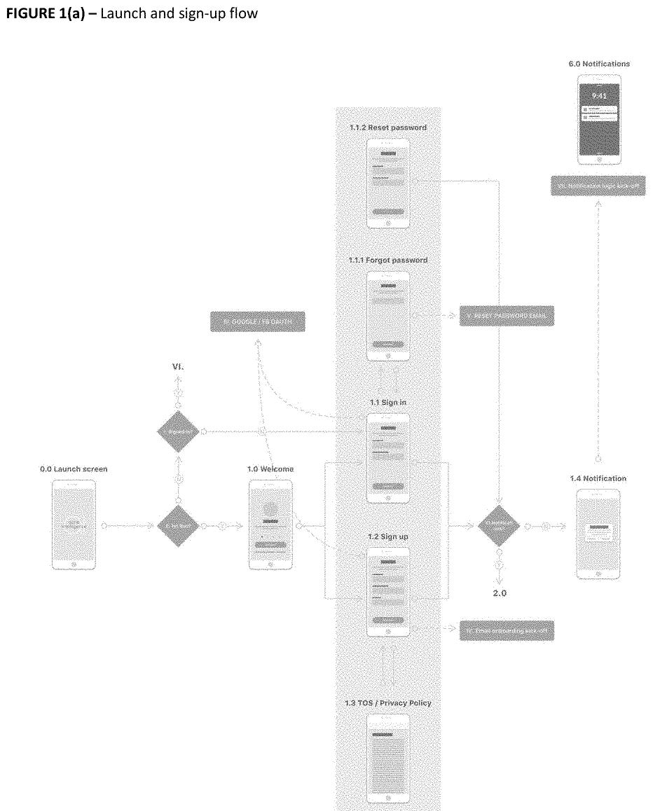

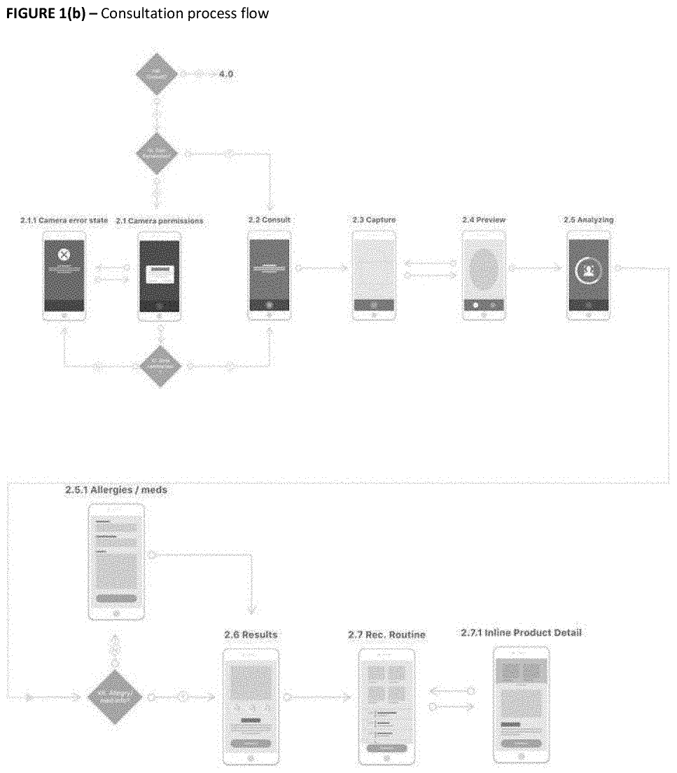

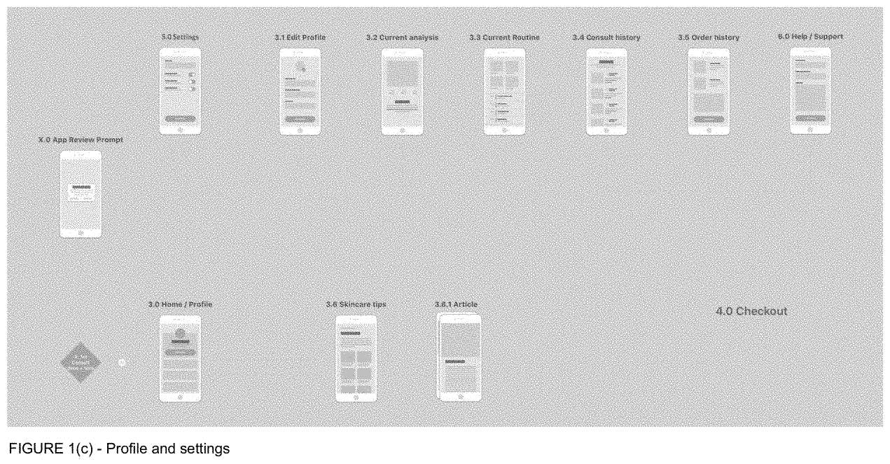

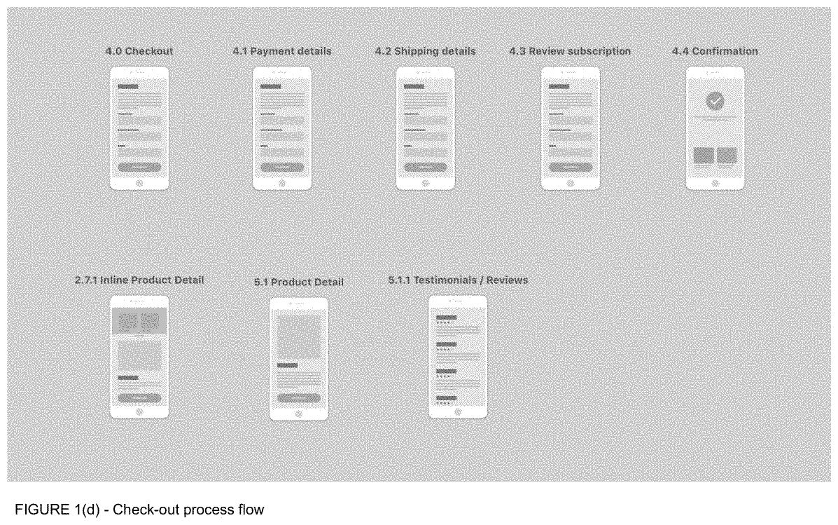

Exemplary steps after app download will be described in the following paragraphs. The general sequence and flow of screens for the app are represented in FIGS. 1 and 1(a)-(d). The screen shots and overall flow are exemplary, and the invention is not meant to be limited to the exact sequence shown.

Once downloaded and opened, the app displays a welcome screen followed by a series of introductory “sliders” with general explanations of the main steps required to obtain a skin analysis. These slides can be viewed one at a time, by sliding each one of them leftwards. There is also an option to skip this introduction (which is only displayed for first time users) by tapping on the “Get started” button at the bottom of each screen.

Following the introductory sliders, the user will register by providing a valid/active email address and selecting a password in the field specifically designated and marked for this purpose. The app will also provide a “social authentication” option where users can register by using their Google or Facebook account credentials. If selecting to register by providing email/password, these are submitted by tapping on the “Create Account” button at the bottom of the screen. Upon submission of email/password, users receive an email notification indicating the account has been created.

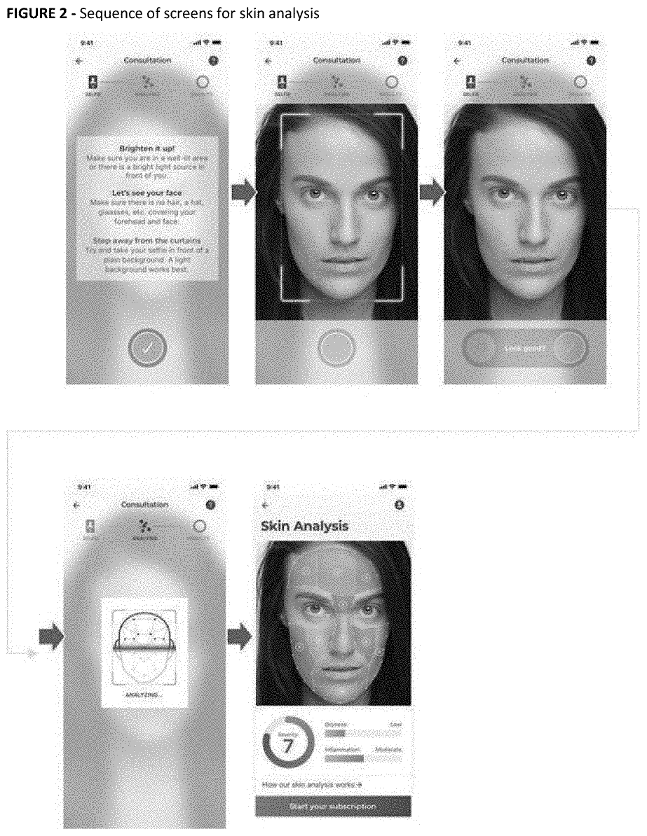

The app will then prompt the user for permissions to be enabled, specifically two: camera and notifications. The camera permissions will be used for capturing a photograph of the user's face for analysis, while the notifications allow for important/relevant information to be shared in a timely and visible manner with the user. Both permissions are required, and once provided, the app will automatically enter the consultation module. The following paragraphs describe an exemplary consultation mode in more detail, which is also illustrated in FIG. 2.

The consultation module automatically turns on the camera on the device. On-screen instructions provide real-time feedback on level of lighting, adjusting automatically as the user moves the camera and/or changes the background and lighting conditions. When the lighting conditions are adequate for the photograph to be captured, the button at the bottom of the screen is enabled and the photograph can now be taken by tapping on it. Upon taking the photograph, the image taken is displayed and user is prompted to confirm if they are satisfied with the image before it is submitted for analysis. Once the image is accepted and submitted, the skin analysis phase begins.

During the skin analysis phase, the user will see an animation of a wireframe in the shape of a face being scanned by a horizontal beam that moves up and down for a few seconds while the analysis is being performed.

At the same time, the photograph is sent to a server where the analysis engine will perform the skin analysis. The engine first applies a set of standardized filters to prepare the image for analysis. Once this is done, the computer vision algorithms will scan and analyze the entire surface. The A.I. has been trained on tens of thousands of images, with a focus on using a diverse range of examples. Images are analyzed with a typical resolution of 0.35 mm to ensure fine details of the skin are visible.

The computer vision algorithms determine the levels of three key variables which are described as follows:

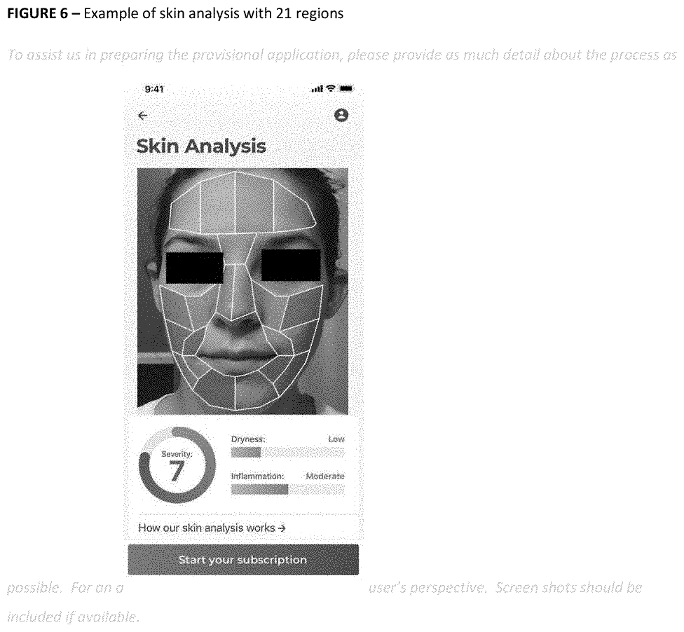

Acne severity: this variable takes into account both the size and number of lesions. Regions with large and numerous lesions are considered to be higher severity. The app will measure from 1 to 10 where 1 is clear skin and 10 is the highest severity: 1=Clear, 2 to 4=Mild, 5 to 7=Moderate and 8 to 10=Severe. The app will obtain multiple measurements (e.g., 21 measurements) based on areas of the face as defined in FIG. 6 but emphasize highest severity in the results and use it for product recommendation

Dryness: Indications of dry skin are increased roughness, skin tone unevenness and redness. The app will measure this variable for the multiple areas in the face as shown on FIG. 6. The result will be displayed as low (1), medium (2 or 3) or high (4 or 5) based on the highest of the multiple areas.

Inflammation: Acne lesions that present with redness and whiteheads are considered by the A.I. as inflammatory acne. By default, the app measures this variable for the multiple areas shown in FIG. 6 and use the highest of the measurements to report in the app results and for product recommendation. The app has the following three levels for this variable: low (Non-Inflamed), medium (Combination of Inflamed and Non-Inflamed) and high (Inflamed).

Beyond the computer vision facial analysis, at the core of the app lies the proprietary product recommendation algorithm. In essence, the algorithm is designed to account for every possible combination of results as measured in the three variables described above and assigns a combination of products that is specific and most adequate for the user's current skin conditions. Moreover, the output of recommended products is also influenced by one additional variable: cycles. In other words, the same combination of results might suggest a certain group of products, but that identical combination of results can suggest a different set of products if a user is in the first cycle versus subsequent cycles of the treatment. This combination of customization factors is a key differentiation point compared to other one-size-fits-all systems, but most importantly it allows for more effective and longer lasting results. Moreover, the line of products behind this algorithm is a state-of-the-art acne line with the latest ingredients and proprietary delivery mechanisms. The line includes various OTC grade SKUs, meaning these have FDA approved levels of active ingredients and are produced under rigorous manufacturing and testing requirements. The algorithm can best be described in six sections, grouped into product types:

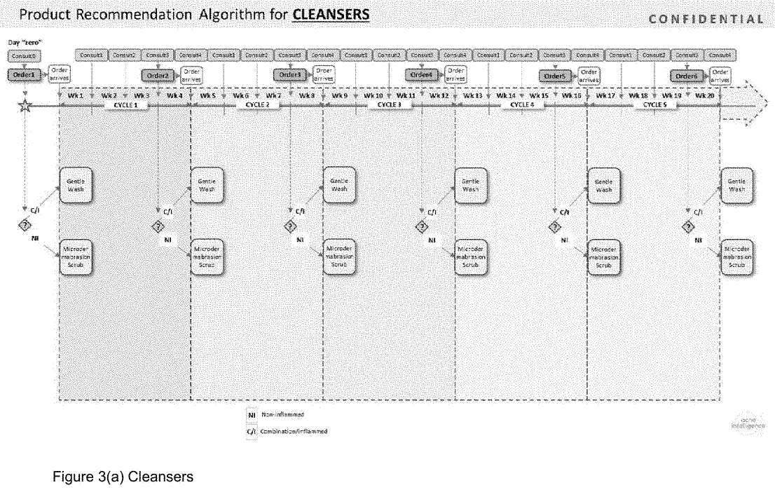

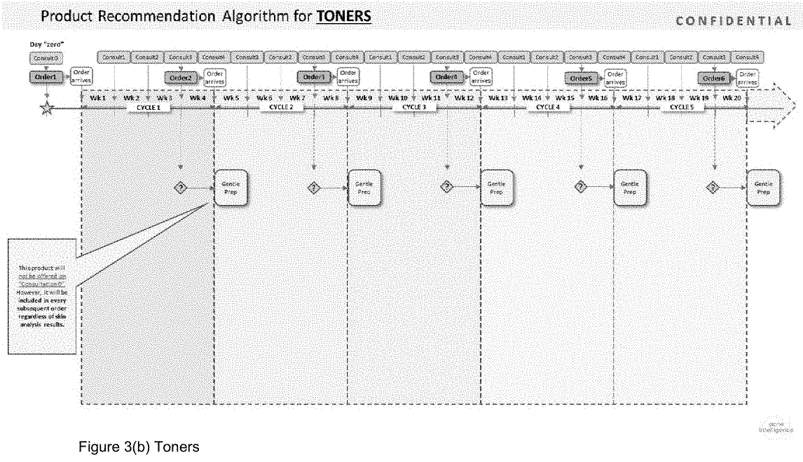

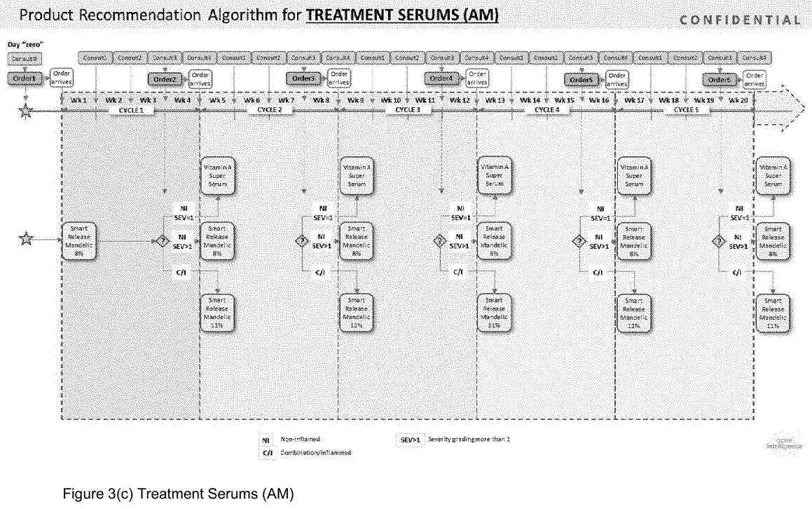

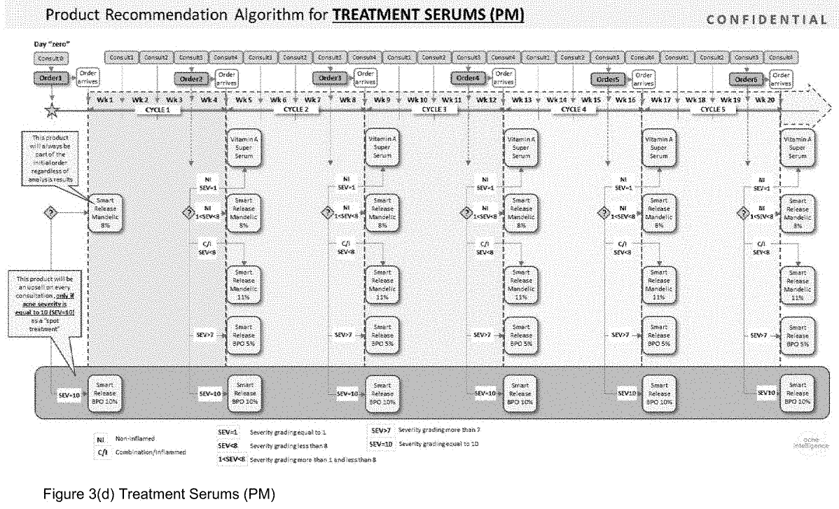

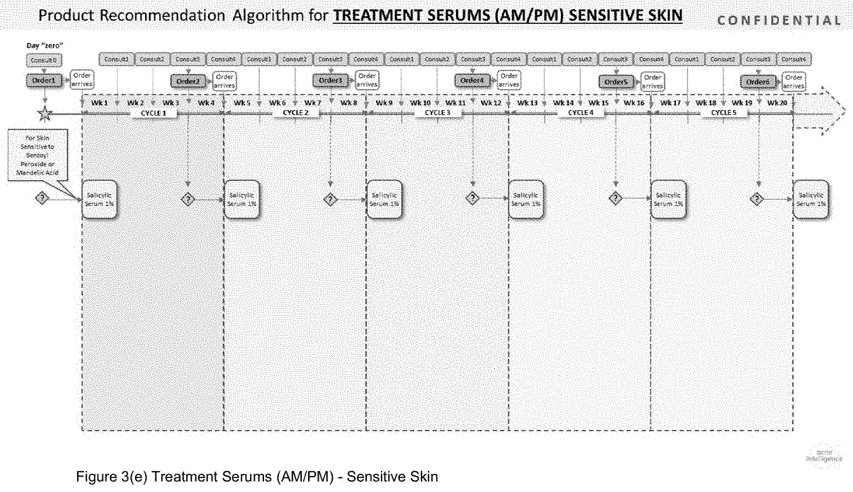

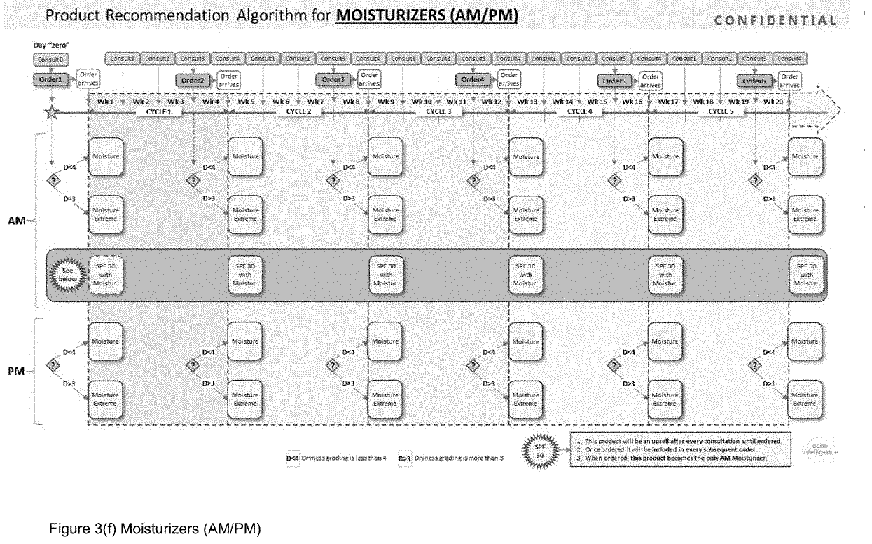

Cleansers (FIG. 3a): This category of products relies on the level of inflammation measured and can results in one of two SKU recommendation. For Combination or Inflamed skin, the Gentle Wash product is recommended while a Non-Inflamed result will suggest the use of the Microdermabrasion Scrub.

Toners (FIG. 3b): In the case of this category, after the first cycle, all users regardless of their skin condition will be recommended to use this product.

Treatment Serums AM (FIG. 3c): The product recommendation algorithm relies on inflammation as well as acne severity results in order to determine which products to suggest. On the first cycle, all users start with Smart Release Mandelic 8%. On the second cycle and onward, there are three variations: Non-Inflamed with acne severity 1 will be recommended Vitamin A Super Serum since this is the mildest treatment which helps both as maintenance and prevention. For Non-Inflamed skin with acne severity of 2 or greater, the Smart Release Mandelic 8% is the optimal recommendation while Combination or Inflamed skin will be recommended the strongest level of treatment, Smart Release Mandelic 11%.

Treatment Serums PM (FIG. 3d): Since an important part of the treatment and healing happens during night time, this is the most complex portion of the algorithm. Similar to Treatment Serums AM, all users regardless of skin condition will start with Smart Release Mandelic 8% in their first cycle. In subsequent cycles, the following 4 alternatives exist: Non-Inflamed with acne severity 1 will be recommended Vitamin A Super Serum, for Non-Inflamed skin with acne severity lower than 8 Smart Release Mandelic 8% is recommended, for Combination/Inflamed with acne severity lower than 8 the Smart Release Mandelic 11% is recommended and finally, all results with acne severity greater than 7 will be recommended Smart Release BPO 5%. In addition to the 4 alternatives measured above, the Smart Release BPO 10% will be an upsell on every consultation I acne severity is equal to 10 as a “spot treatment”.

Treatment Serums AM/PM Sensitive Skin (FIG. 3e): For skins that are known to be sensitive or allergic to the active ingredients in the other treatment serums (as determined by a few simple questions during sign-up), the algorithm defaults (both AM and PM) to recommend Salicylic Serum 1%. This product is specifically designed and formulated to treat acne for sensitive skin.

Moisturizers AM/PM (FIG. 3f): In this category, the product recommendation algorithm relies on the dryness variable to determine optimal product recommendations. If the skin's dryness level is lower than 4, Moisture is recommended. For dryness levels higher than 3, Moisture Extreme is recommended. This is the same for all cycles and also the same for AM and PM routines. The only variation for AM is that SPF 30 with Moisturizer is recommended as an upsell initially and once purchased, it is included in every subsequent set of recommended products to be used as an AM moisturizer.

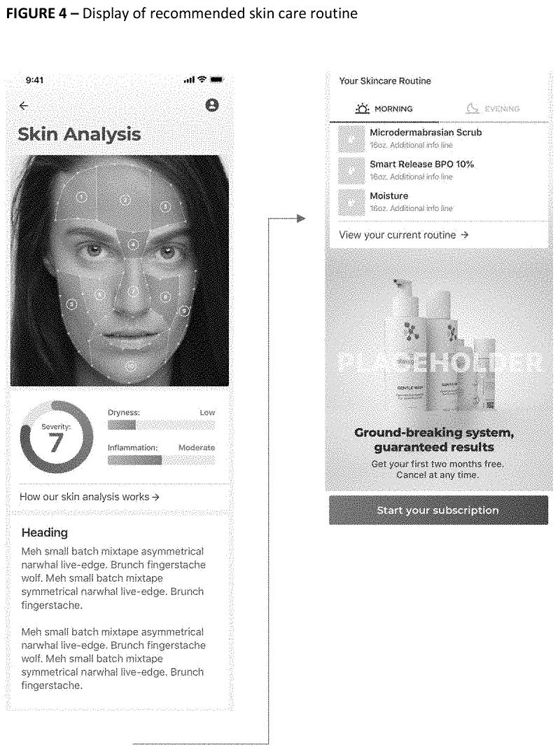

The culmination of the process is represented in the Skin Analysis screen (FIG. 4), which graphically displays the results from the computer vision analysis as well as the results from the product recommendation algorithm.

First, there is the image of the user's face as captured by the smart phone camera for analysis, with an overlay of a wireframe that divides the face into multiple regions (e.g., 21 regions). These regions are shaded in tones of red, based on acne severity results for each region 1=no shading, 2 to 4=50% red, 5 to 7=75% red and 8 to 10=100% RED. After the photograph with the overlay, the results of the skin analysis for the three variables are displayed graphically (and numerically as well for acne severity). This display of results is followed by a paragraph that summarizes them, which is dynamic, and changes based on the variety of result combinations. The recommended products are first displayed in the form of the routines. Starting with AM routine, the recommended products to be used in the morning are displayed and a tab is available to switch this display to the recommended products for the PM routine. The bottom of this screen displays a call to action, suggesting the user signs-up for the subscription to get the recommended products shipped and continue the process cycle after cycle.

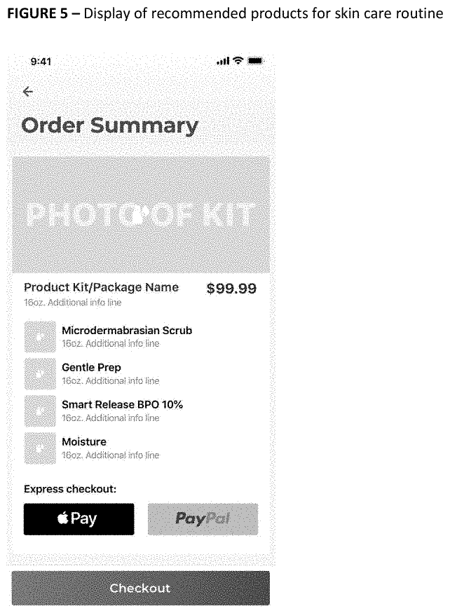

Upon confirming subscription, a summary screen (FIG. 5) is displayed with the shopping cart and listed products/prices as recommended by the analysis/algorithm. Subsequently, standard shipping/billing information is captured to conclude the checkout process and stored for future cycles.

This consultation process is repeated multiple times within the context of each “cycle”. Cycles may have a 4-week duration, for example, and there is no predetermined/limited number of cycles; customers use certain products to get clear of acne and others to stay clear as defined by the consultations and the algorithm.

Customers receive a notification to conduct a consultation every week. A consultation should take less than a minute and is done through the app as described in previous paragraphs. With a 4-week cycle, although the consultation is conducted on a weekly basis, on the third week of every cycle, based on the combination of the results and a predefined algorithm, the app indicates which products need to be used in the next 4-week cycle.

As a consequence of the above, with the exemplary 4-week cycle, products are shipped every four weeks based on consultation results. The order gets placed at the end of the third week of each cycle (following the consultation) and takes a week or less to arrive so that they are available before the beginning of the next cycle.

All products will be used in the morning (AM) and/or at night (PM). The sequence and instructions for using the products will be grouped in routines, one AM and one PM. These routines will be easily accessible in the app and also as an option through notifications in the app at predetermined (user defined) times every day.

The app recommends and prompts (via notifications) to perform an analysis every week. Within the four-week cycles, for example, upon completing the third week analysis in each cycle, the order is placed based on the results of that third week analysis. This ensures product availability (considering shipping/transit times) for the beginning of the next cycle. If the third week analysis is not performed, the app will utilize the results from the most recent analysis available for the user.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of diagnosing and treating a skin condition comprising:
(a) registering a user via a user mobile device in communication with a system server;
(b) prompting the user to enable camera and notification permissions;
(c) directing the user to capture a facial image;
(d) the system server receiving the facial image and running software via an analysis engine to perform a skin analysis; and
(e) the system server assigning one or more products and a treatment protocol based on the skin analysis.

2. A method according to claim 1, wherein step (d) comprises determining levels of skin condition severity for acne severity, dryness and inflammation.
